(19) United States
(12) Patent Application Publication
(10) Pub. No.: US 2024/0183080 A1
(43) Pub. Date: Jun. 6, 2024

(54) PRODUCING A COMBINED MATRIX AND CELLS IN SITU ON A DAMAGED TISSUE AND APPARATUS FOR IMPLEMENTING THE SAME

(71) Applicant: NANOMEDIC TECHNOLOGIES LTD., Lod (IL)

(72) Inventors: Claudia Monica BARZILAY, Modiin Macabim Reut (IL); Chen BARAK, Shoam (IL); Mark DARTY, Collierville, TN (US)

(21) Appl. No.: 18/435,004

(22) Filed: Feb. 7, 2024

Related U.S. Application Data
(63) Continuation of application No. PCT/IL2022/050863, filed on Aug. 8, 2022.
(60) Provisional application No. 63/230,756, filed on Aug. 8, 2021.

(51) Int. Cl. D01D 5/00; A61L 27/54; A61L 27/56

(52) U.S. Cl. CPC D01D 5/0084; A61L 27/54; A61L 27/56; D01D 5/0038

(57) ABSTRACT

A method of producing a matrix in situ on a damaged tissue; said method comprising steps of:
producing a fibrous scaffolding mat by means of electrospinning a fiber-containing medium on said damaged tissue;
   applying a bioactive material to said damaged tissue; said bioactive material is selected from the group consisting of human cells, stem cells, manipulated cells, collagen, gelatin, blood or blood components and any combination thereof.

### CROSS REFERENCE TO RELATED APPLICATIONS

This application is a Bypass Continuation of PCT Patent Application No. PCT/IL2022/050863 having International filing date of Aug. 8, 2022, which claims the benefit of priority of U.S. Provisional Patent Application No. 63/230,756, filed Aug. 8, 2021, the contents of which are all incorporated herein by reference in their entirety.

### FIELD OF THE INVENTION

The present invention relates to methods and devices for treatment of damaged tissues and more particularly, to electrospinning fibrous mats combined with applying bioactive materials in an in-situ manner.

### BACKGROUND OF THE INVENTION

Wounds (related to surgery, trauma, diabetes, pressure, poor venous or arterial flow, burns, etc.) and compromised wound healing are major concerns for the public health sector; they cause significant discomfort, pain and stress while draining the medical system of an enormous amount of resources.

Complex and lengthy treatments cause an increasing burden on healthcare expenses. Burns, chronic and other difficult to treat wounds often require surgery and extended hospitalization periods. In the United States (US) alone, millions of patients need treatment for chronic wounds and an estimated US $50 billion is spent annually (data published by ‘Wound Care Awareness Week-2017’, including direct and indirect costs). Things do not go better in Europe: in the United Kingdom (UK), about 200,000 patients have a chronic wound and in the European Community the prevalence is expected over 1.5 million people. Indeed, a proper and faster healing process is cost-saving, reduces or avoids health care provider visits, onset of infections and hospitalization while improving quality of life and ability to return to work for patients. Therefore, hard to heal and complex wounds' management is a strategic objective for National Health Systems.

Electrospinning is a unique technique that uses electrostatic forces to produce nano-diameter, non-woven fibers that incorporate very fine pore sizes with high surface area, making them an ideal solution for delivering wound healing therapies to any surface of the body. Many types of polymers can be electrospun creating a bio-polymeric product able to contain wound healing agents such as antibacterial, silicon, collagen and cells. The integration of electrospinning technology into a portable, bedside device, offers immediate in-situ wound care treatment, creating a fully personalized customized nano-fibrous skin substitute matrix/layer based on patient's wound condition, size and contour.

Electrospinning technology uses electric forces to generate on the spot, nanofibrous matrices or skin-substitute layers which facilitate and enhance the body healing process. The matrix/layer obtained is fine-tunable to surface, shape, thickness, skin site and is microscopically adherent to all body surfaces. The use of the in-situ portable apparatus as a delivery system for cells, micrografts, autologous cells, cultured epithelial autograft (CEA), stem cells etc, combined with the nanofibrous matrix and skin substitute electrospun layer either as separate phases or as a directly combined cell suspension with a polymer solution in a one-step application will enhance and accelerate wound healing, cell proliferation and regeneration. This combined product will revolutionize the regenerative treatment option replacing long healing processes and surgical procedures.

Cells, micrografts, autologous cells, cultured epithelial autograft (CEA), stem cells etc are widely discussed in the scientific literature as being able to maintain a high regenerative potential and used in non-healing wounds, acute or chronic, post-surgical wounds, post-traumatic wounds, vascular or diabetic ulcers, burns, etc. From the clinical-practical point of view, this technology allows the donor tissue collected from the patient to be significantly smaller than the size of the target wound, while being minimally invasive for the patient. In fact, the harvest, processing and implantation of the cells can take place at the same surgical or in a dedicated skin laboratory.

Electrospinning is a nanofiber production method which uses electric force to draw threads of polymer up to fiber diameters in the order of few hundred nanometers. The use of the portable electrospinning apparatus, minimizing the electrospinning technology from large machine into a hand-held, battery-operated device, allows to integrate the electrospinning technology into a bedside device that offers immediate in-situ wound care treatment by generating a skin substitute matrix/layer facilitating the body healing process. It enhances the inherent characteristics of the electrospun nanofibers, mimicking the structure of the extracellular matrix and body tissue and thus providing an excellent scaffold for tissue integration, proliferation and regeneration. The electrospun matrix obtained in situ is fine-tunable to surface, shape, thickness, skin site and area to be covered, microscopically adhering to all body surfaces and allowing early showers. It is applied from a short distance, eliminating contact between the caregiver and the wound, therefore reducing the potential of infection. In addition, it facilitates cell respiration, oxygen permeation and regulation of moisture level while protecting against microbial penetration due to its porous structure.

Thus, there is a long-felt and unmet need to provide methods and apparatuses configured for combining of cells, micrografts, autologous cells, cultured epithelial autograft (CEA), stem cells etc. and the portable electrospinning. Such an apparatus applying the electrospun nanofibrous skin substitute matrix/layer will provide a complete solution for human tissue regeneration, specifically addressing the wound care field but also other areas such as aesthetics and cosmetics. The nanofibrous matrix/layer can be made of different synthetic or biologic compounds that enhance the scaffold function and can be degradable, absorbable or resorbable or can peel off spontaneously as the new skin underneath is fully epithelialized. The polymer solution can be combined and enhanced with various additives according to the nature of the wound and the patient's needs: antibacterial, antibiotics, collagen, silicon, hydrogel, cannabinoids and more.

### SUMMARY OF THE INVENTION

It is hence one object of the invention to disclose a method of producing a matrix in situ on a damaged tissue. The aforesaid method comprises steps of: (a) producing a fibrous scaffolding mat by means of electrospinning a fiber-containing medium on said damaged tissue; (b) applying a active or bioactive material to said damaged tissue; said bioactive material is selected from the group consisting of human cells, stem cells, manipulated cells, collagen, gelatin and any combination, blood or blood components thereof.

Another object of the invention is to disclose the step of applying said bioactive material selected from the group consisting of depositing said bioactive material onto said damaged tissue before said step of said fibrous scaffolding mat, depositing said bioactive material onto an in-situ produced fibrous scaffolding mat, performing said step producing a fibrous scaffolding mat and applying a bioactive material in a concurrent manner and any combination thereof.

A further object of the invention is to disclose the step of applying said bioactive material performed by a technology selected from the group consisting of mechanically spreading said bioactive material, electrospraying said bioactive material and a combination thereof.

A further object of the invention is to disclose the bioactive material derived from a source selected from the group consisting of mammals, fish, reptiles, human donors and any combination thereof.

A further object of the invention is to disclose the fiber-containing medium comprising fibers selected from the group consisting of synthetic fibers, fibers derived from natural environment and a combination thereof.

A further object of the invention is to disclose an apparatus for producing a matrix in situ on a damaged tissue; said apparatus comprising: (a) an electrospinning arrangement configured for electrospinning a fiber-containing medium in situ on a damaged tissue such that a fibrous scaffolding mat is produced; and (b) an applicator configured for applying a bioactive material to said damaged tissue; said active or bioactive material is selected from the group consisting of human cells, stem cells, manipulated cells, collagen, gelatin, blood or blood components and any combination thereof.

A further object of the invention is to disclose the applicator configured for performing an operation selected from the group consisting of depositing said bioactive material onto said damaged tissue before said step of said fibrous scaffolding mat, depositing said bioactive material onto an in-situ produced fibrous scaffolding mat, performing said step producing a fibrous scaffolding mat and applying a bioactive material in a concurrent manner and any combination thereof.

A further object of the invention is to disclose the electrospinning arrangement further comprises a medium container accommodating said electrospinning medium to be electrospun, and an electrospinning nozzle in fluid communication with said container configured for exhausting said electrospinning medium therefrom.

A further object of the invention is to disclose the applicator comprising a material container accommodating said bioactive material to be electrosprayed and an electrospraying nozzle configured for exhausting said bioactive material therefrom.

A further object of the invention is to disclose the medium container and material container forming an integral compartmentalized body.

A further object of the invention is to disclose the medium container and material container which are arranged coaxially to each other.

A further object of the invention is to disclose the medium container and material container which are consecutively brought into fluid communication with a common nozzle such that said electrospinning medium and bioactive material are exhausted from said common nozzle in a consecutive manner.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In order to understand the invention and to see how it may be implemented in practice, a plurality of embodiments is adapted to now be described, by way of non-limiting example only, with reference to the accompanying drawings, in which

FIG. 1 is a flowchart of a method of producing a matrix in situ on a damaged tissue;

FIGS. 2 to 5 are schematic diagrams of alternative embodiments of an apparatus for producing a matrix in situ on a damaged tissue;

FIGS. 6a and 6b are graphs illustrating viability of keratinocytes deposited by electrospraying;

FIGS. 7 and 8 present photographs illustrating an effect of in-situ electrospun fibers scaffold on keratinocyte growth with feeder and without it, respectively; and

FIG. 9 presents photographs illustrating an effect of in-situ electrospun fibers scaffold on fibroblast growth.

### DETAILED DESCRIPTION OF THE INVENTION

The following description is provided, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide a method of producing a matrix in situ on a damaged tissue and an apparatus for implementing the same.

The use of the portable electrospinning apparatus as a delivery system for the cells combined with the nanofibrous skin substitute matrix/layer either as separate phases or as a directly combined cell suspension with a polymer solution in a one-step application, will enhance and accelerate wound healing and cell proliferation and regeneration while increasing the efficiency of this regenerative treatment option.

The use of the portable electrospinning device as a delivery method for the cells involves a unique mode of operation, often referred to as electrospray. Electrospray is an electrohydrodynamic technique similar to electrospinning. It is governed by similar principle and uses identical apparatus, that is, a high-voltage power supply and a syringe filled with a precursor solution. During electrospray, a stable Taylor cone is also formed, which is stabilized by the liquid surface tension, electrostatic force and gravity. Compared to electrospinning, the degree of electrostatic stretch over the surface tension is relatively low during electrospray, leading to the formation of particulate products (nanoparticles or microparticles) instead of fibrous products. Electrospray can be simple electrospray and coaxial electrospray, with the use of different types of spinneret (either a simple spinneret or a coaxial spinneret) and usually involves two immiscible liquids merged out of spinneret to form conically shaped cone-jet. Supporting the delivery of the cells combined with the nanofibrous skin substitute matrix/layer either as separate phases or as a directly combined cell suspension with a polymer solution in a one-step application. The high versatility in fabricating microparticles with tunable structures, demonstrates a great potential for drug delivery applications as well.

Keratinocytes from two different human sources (A keratinocytes and B keratinocytes) were seeded directly (control cells) or by cell electrospray on tissue dishes coated with or without i3T3-J2 feeder cells. After 3 days the viability was tested by three different assays: automatic cell counter, cell count and FACS.

The term “bioactive material” hereinafter equally refers to the materials derived from the natural environment, to materials having a biologic source and to synthetic materials.

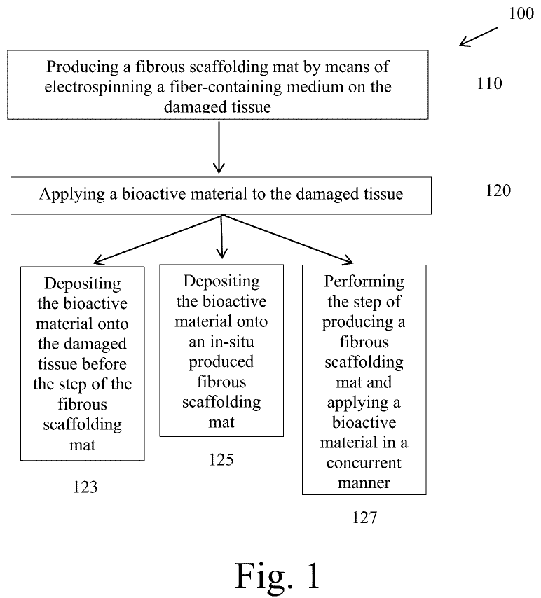

Reference is now made to FIG. 1 presenting a flowchart of method 100 directed to producing a matrix in situ on a damaged tissue. Method 100 comprises two steps of producing a fibrous scaffolding mat by means of electrospinning a fiber-containing medium on the damaged tissue (step 110) and applying a bioactive material to the damaged tissue (step 120). Method 100 can be alternatively embodied as follows. The bioactive material is deposited onto the damaged tissue before forming the fibrous scaffolding mat thereon (step 123); the bioactive material is deposited onto the in-situ produced fibrous scaffolding mat (step 125); and applying the bioactive material (step 120) and electrospinning the fiber-containing medium (step 110) are performed concurrently. Step 120 can be performed by means of mechanically spreading the bioactive material manually or electrospraying the aforesaid material over a target area.

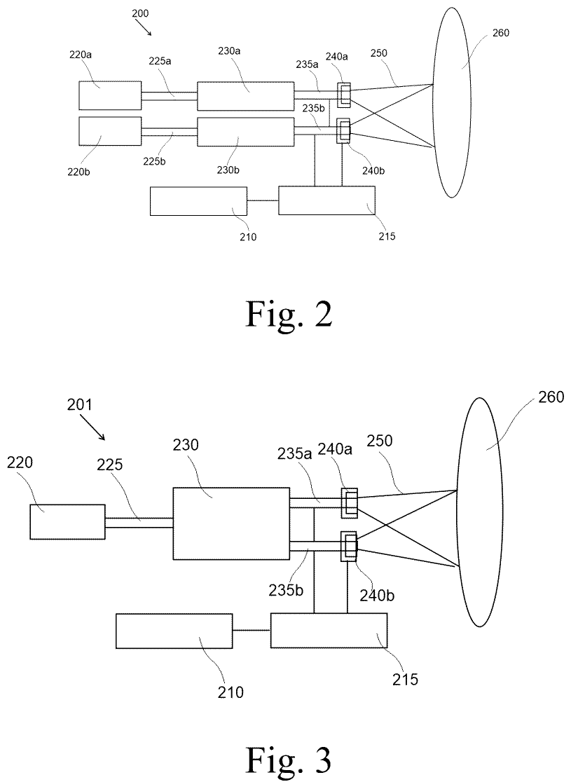

Reference is now made to FIG. 2 presenting a schematic diagram of apparatus 200 for multimodal therapy. Numeral 260 refers to a target area of the damaged tissue. Apparatus 200 comprises battery or low-voltage power supply 210 energizing high-voltage power supply 215. Difference of electric potentials generated by high-voltage power supply 215 is applied between connected in parallel nozzles 235a/235b and auxiliary electrodes 240a/240b. Lines 250 schematically show trajectories of electrospun and electrosprayed material beams. The fiber-containing medium and bioactive material are electrospun and electrosprayed via auxiliary electrodes 240a and 240b, respectively. Container 230a accommodates the fiber-containing medium which is dispensed from container 230a by dispensing means 225a such as a piston movable by motor 220a. The bioactive material accommodated in container 230b is dispensed by dispensing means 225b by motor 220b.

It should be that all the treatment protocols described above are implementable by apparatus 200 because the parts dedicated for electrospinning and electrospraying are independent from each other. According to one embodiment of the present invention, the apparatus is controlled by a microcontroller (not shown) preprogrammed for implementing a number of predetermined protocols.

Reference is now made to FIG. 3 presenting alternative embodiment 201 of the present invention. Each container which can accommodate the materials to be electrospun and electrosprayed can have more than 1 nozzle (for example, 240a and 240b). Analogously to the previous embodiment 200, the material accommodated in container 230 is dispensed by means 225 moved by motor 220.

Reference is now made to FIG. 4 presenting alternative embodiment 203 of the present invention. Container 230c (possibly commercially available cartridge) is longitudinally compartmentalized such that the materials to be electrospun and electrosprayed are accommodated in compartments arranged in parallel to each other. Independence of dispensing means 225a/225b and motors 220a/220b allows any protocol of the treatment.

Reference is now made to FIG. 5 presenting alternative embodiment 203 of the present invention. Container 230d is transversely compartmentalized such that the materials to be electrospun and electrosprayed are accommodated in compartments disposed in series in a coaxial manner. Both materials are successively dispensed by dispensing means 225 moved by motor 220.

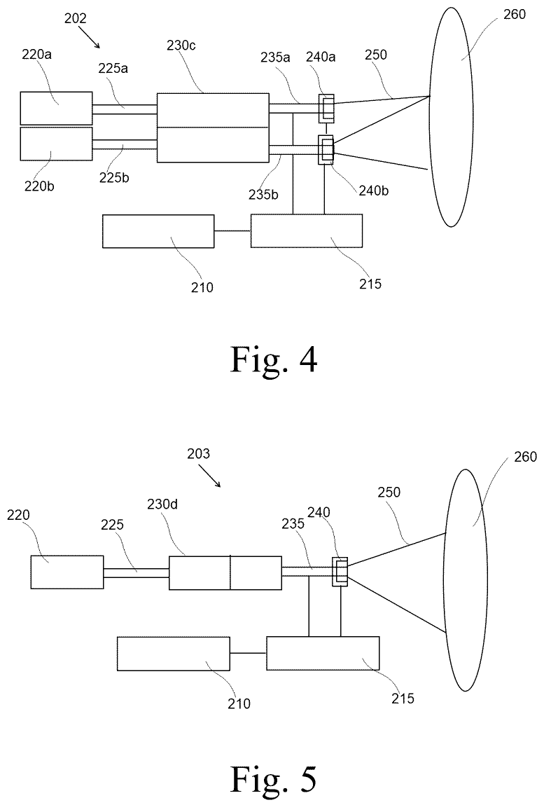

Reference is now made FIGS. 6a and 6b presenting graphs illustrating viability of keratinocytes deposited by electrospraying. It is experimentally shown that electrospraying keratinocytes cells has no significant effect on their viability in comparison to control samples.

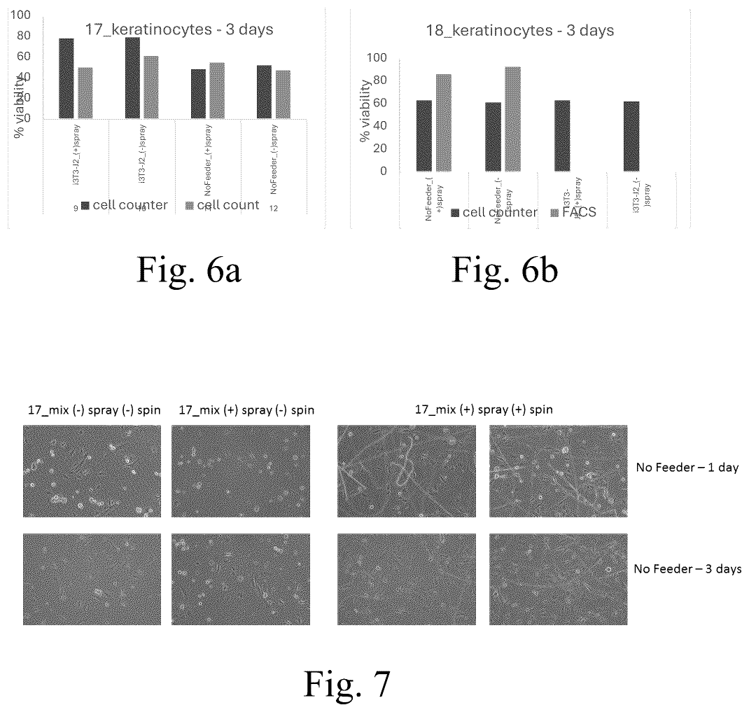

Reference is now made FIG. 7 presenting photographs of culture dishes coated with in-situ electrospun fibers scaffold. It is experimentally shown that the electrospun fibers scaffold facilitates growth of keratinocytes in culture in the absence of a feeder layer. Keratinocytes which were seeded directly or by cell-electrospraying on tissue dishes coated with or without Spincare electrospun fibers. After 1 day (upper panel) and 3 days (lower panel) cells were photographed by inverted microscope. It should be noted that the keratinocytes have very low adherence to the culture plate without the presence of feeder layer cells nor the in-situ electrospun fibers scaffold. On the other hand, the in-situ electrospinning scaffold enables the keratinocytes to adhere to the culture plate and to successfully proliferate and grow in culture.

Reference is now made FIG. 8, presenting photographs of keratinocyte cultures which were seeded directly or by cell electrospray on i3T3-J2 feeder cells coated with or without the in-situ electrospun scaffold. After 1 day (upper panel in FIG. 3) and 3 days (lower panel in FIG. 3) the cells were photographed by inverted microscope. The keratinocytes showed enhanced proliferation and growth in culture in presence of the in-situ electrospun fibers against the control (only feeder without electrospun scaffold).

Following examination of a mixture of fibroblasts and keratinocytes cells shows ability of growth and proliferation in culture on in-situ electrospun scaffold without a supporting feeder layer. Growth of keratinocytes cells, fibroblasts cells (1×10⁵ each), and a 1:2 mixture of them were tested for cell viability and assessed by standard XTT test.

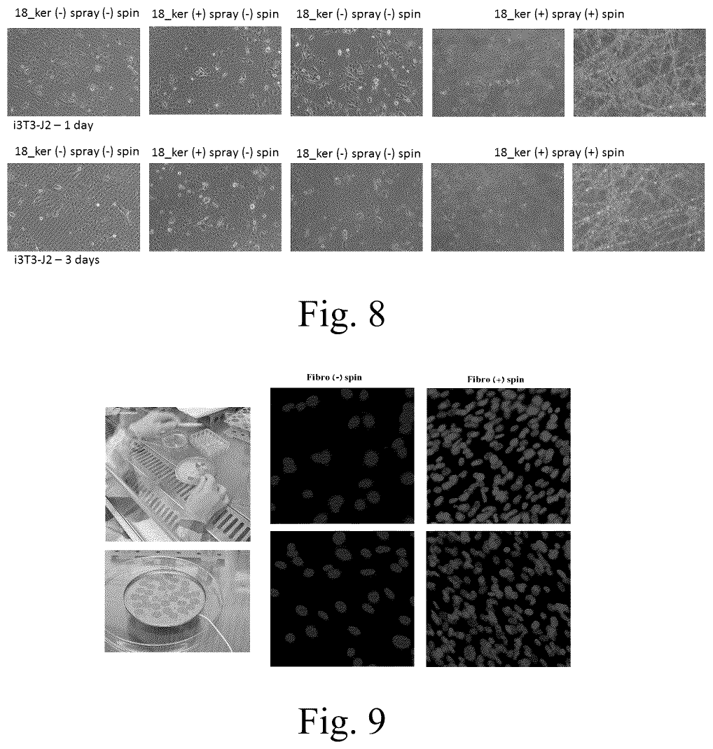

Reference is now made FIG. 9, presenting photographs of experimental cultures grown on in-situ electrospun fibers and without them. Significant growth enhancement on the in-situ electrospun fibers as scaffold is experimentally obtained.

This unique and combined approach integrates cells and nanofibrous electrospun matrix/layer in one procedure, on the spot, in-situ or with combination of cells from skin laboratories/tissue banks to enhance the efficiency of both delivery and uptake, proliferation, regeneration of the cells to achieve excellent tissue regeneration and healing processes.

The realization of this product could have a big impact in the field of wound care both improving the quality of life of the patients and reducing economic burden in terms of the number of follow up visits, additional treatments often needed in order to manage complex and hard to heal wounds.

While the invention has been particularly shown and described with reference to an embodiment thereof, it will be appreciated by those skilled in the art that various changes in form and detail may be made without departing from the spirit and scope of the invention.

1.-15. (canceled)

16. A method of applying bioactive material in situ to a damaged tissue; said method comprising
producing a fibrous scaffolding mat by means of electrospinning a medium containing a fiber and/or polymer on said damaged tissue; and
electrospraying said bioactive material on said damaged tissue, said bioactive material being selected from the group consisting of cells, stem cells, manipulated cells, polymer, blood, blood components, and any combination thereof.

17. The method according to claim 16, wherein said bioactive material is derived from a source selected from the group consisting of mammals, fish, reptiles, human, and any combination thereof.

18. The method according to claim 16, wherein said fiber and/or polymer is selected from the group consisting of synthetic fibers and/or polymer, fibers and/or polymer derived from natural environment, and a combination thereof.

19. The method according to claim 16, wherein said polymer comprises collagen and/or gelatin.

20. The method according to claim 16, wherein applying said bioactive material comprises electrospraying said bioactive material onto said damaged tissue before producing said fibrous scaffolding mat.

21. The method according to claim 16, wherein said applying bioactive material comprises electrospraying said bioactive material onto an in-situ produced fibrous scaffolding mat.

22. The method according to claim 16, wherein said applying bioactive material comprises producing said fibrous scaffolding mat and electrospraying said bioactive material in a concurrent manner.

23. A portable apparatus for applying bioactive material in situ on a damaged tissue, said apparatus comprising
an electrospinning arrangement configured for electrospinning a medium comprising a fiber and/or a polymer in situ on a damaged tissue such that a fibrous scaffolding mat is produced, the electrospinning arrangement comprising a medium container configured for accommodating the medium; and
an electrospraying arrangement configured for electrospraying a bioactive material selected from the group consisting of cells, stem cells, manipulated cells, polymer, blood or blood components, and any combination thereof, on the damaged tissue, the electrospraying arrangement comprising a material container configured for accommodating the bioactive material.

24. The portable apparatus according to claim 23, wherein said bioactive material is derived from a source selected from the group consisting of mammals, fish, reptiles, human, and any combination thereof.

25. The portable apparatus according to claim 23, wherein said fiber-containing medium comprises fibers selected from the group consisting of synthetic fibers, fibers derived from natural environment and a combination thereof.

26. The portable apparatus according to claim 23, wherein said electrospraying arrangement further comprises an electrospraying nozzle configured for exhausting said bioactive material therefrom.

27. The portable apparatus according to claim 23, wherein said electrospinning arrangement further comprises an electrospinning nozzle in fluid communication with said container configured for exhausting said electrospinning medium therefrom.

28. The portable apparatus according to claim 23, wherein said medium container and material container form an integral compartmentalized body.

29. The apparatus according to claim 28, wherein said medium container and material container are arranged coaxially to each other.

30. The portable apparatus according to claim 23, wherein said medium container and material container are consecutively brought into fluid communication with a common nozzle such that said electrospinning medium and bioactive material are exhausted from said common nozzle in a consecutive manner.

31. The portable apparatus according to claim 23, wherein said medium container and material container are brought into fluid communication with a common nozzle such that said electrospinning medium and bioactive material are concurrently exhausted from said common nozzle.

32. The portable apparatus according to claim 23, further comprising a battery.
